(19) United States
(12) Patent Application Publication
(10) Pub. No.: US 2023/0292856 A1
(43) Pub. Date: Sep. 21, 2023

(54) FACE MASK WITH MOISTURE LEVEL AND OXYGEN SATURATION DETECTION

(71) Applicant: Change Healthcare Holdings, LLC, Nashville, TN (US)

(72) Inventor: David Alan Wright, Nashville, TN (US)

(21) Appl. No.: 17/697,142

(22) Filed: Mar. 17, 2022

(51) Int. Cl. A41D 13/11; A61B 5/097; A61B 5/093; A61B 5/00

(52) U.S. Cl. CPC A41D 13/11; A61B 5/097; A61B 5/0935; A61B 5/0004; A61B 5/0022; A61B 5/746; A61B 5/742; A61B 2562/164; A61B 2562/029

(57) ABSTRACT

A face mask includes a mask body formed of a flexible material and configured to cover the mouth and nose of the user such that a cavity is formed between the face of the user and the face mask, the mask body having an interior surface facing the mouth and nose of the user and an exterior surface opposite the interior surface; a first sensor unit for measuring a moisture level and an oxygen level of the air entering the cavity; a second sensor unit for measuring a moisture level and an oxygen level of the air being expelled from the cavity; and at least one wireless transceiver configured to transmit data from the first sensor unit and the second sensor unit to a remote device.

### BACKGROUND

The present disclosure relates generally to face masks and more specifically to face masks that are intended to be worn by a user for protection against airborne pathogens.

Face masks have long been used in a variety of industries to protect users from airborne dust, debris, bacteria, viruses, and the like. Medical professionals, for example, often wear face masks (e.g., N95, surgical, etc.) to reduce the spread of harmful diseases, which can help to protect both the medical professionals themselves and their patients. In more recent years, the COVID-19 pandemic has illustrated an increasing need for consumer face masks that offer adequate protection against bacteria and viruses, such as COVID-19. Many users have turned to commercially-available KN95, surgical-style, or even cloth face masks. While these types of masks do offer a level of protection to a wearer and/or those in proximity to the wearer, they face a number of drawbacks. For example, KN95 and surgical-style masks are often single-use, which creates waste and increasing costs for the wearer. Cloth masks are a favorable reusable alternative; however, cloth mask can because saturated with moisture over a period of time (e.g., due to the wearer's breath) which can greatly reduce their effectiveness. Further, none of these commonly-available masks offer any sort of feedback to a user regarding moisture and oxygen levels, which could help to reassure the wearer that the mask is operating effectively.

### SUMMARY

One implementation of the present disclosure is a face mask configured to be worn by a user. The face mask includes a mask body formed of a flexible material and configured to cover the mouth and nose of the user such that a cavity is formed between the face of the user and the face mask, the mask body having an interior surface facing the mouth and nose of the user and an exterior surface opposite the interior surface, a first sensor unit for measuring a moisture level and an oxygen level of the air entering the cavity, a second sensor unit for measuring a moisture level and an oxygen level of the air being expelled from the cavity, and at least one wireless transceiver configured to transmit data from the first sensor unit and the second sensor unit to a remote device.

In some embodiments, the face mask further includes at least one light emitting diode (LED) positioned on one of the mask body, the first sensor unit, or the second sensor unit, and the at least one LED is configured to illuminate a first color if any of the moisture level of the air entering the cavity, the oxygen level of the air entering the cavity, the moisture level, or the oxygen level of the air being expelled from the cavity exceeds a threshold value or illuminate a second color if each of the moisture level of the air entering the cavity, the oxygen level of the air entering the cavity, the moisture level, and the oxygen level of the air being expelled from the cavity is below the threshold value.

In some embodiments, the first sensor unit is housed in a first valve configured to open when the user inhales to allow air to enter the cavity formed by the face mask, the first valve extending from the interior surface to the exterior surface of the mask body.

In some embodiments, the second sensor unit is housed in a second valve configured to open when the user exhales to expel air from the cavity formed by the face mask, the second valve extending from the interior surface to the exterior surface of the mask body.

In some embodiments, the first sensor unit and the second sensor unit are removably coupled to the mask body such that the first sensor unit and the second sensor unit can be removed to wash the face mask.

In some embodiments, the remote device compares the moisture level and the oxygen level of the air entering the cavity to the moisture level and the oxygen level of the air being expelled from the cavity to determine whether a moisture level of the face mask or the user's oxygen saturation exceeds corresponding threshold values.

In some embodiments, the at least one wireless transceiver includes a short-range, ultra-high frequency (UHF) transceiver.

In some embodiments, the flexible material includes a washable, non-woven fabric or a plurality of washable, woven fabric layers.

Another implementation of the present disclosure is a method including receiving first data from a first sensor positioned on a face mask configured to be worn by a user, the first sensor unit measuring a moisture level and an oxygen level of the air entering a cavity formed between the face of the user and the face mask as the user inhales, receiving second data from a second sensor unit that measures a moisture level and an oxygen level of the air being expelled from the cavity as the user exhales, comparing the first data and the second data to determine an oxygen saturation for the user and an amount of moisture retained by the face mask, and providing a notification to the user if the oxygen saturation of the user exceeds a first threshold or if the amount of moisture retained by the face mask exceeds a second threshold.

In some embodiments, the oxygen saturation for the user is determined based on a difference between the oxygen level of the air entering the cavity and the oxygen level of the air being expelled from the cavity.

In some embodiments, the amount of moisture retained by the face mask is determined based on a difference between the moisture level of the air entering the cavity and the moisture level of the air being expelled from the cavity.

In some embodiments, providing the notification includes at least one of illuminating a light emitting diode (LED) positioned on the face mask or presenting a graphical user interface via a user device associated with the user, the graphical user interface indicating the notification and presenting at least one of the first data or the second data.

In some embodiments, the first sensor is housed in a first valve positioned on the face mask and configured to open when the user inhales to allow air to enter the cavity formed by the face mask.

In some embodiments, the second sensor is housed in a second valve positioned on the face mask and configured to open when the user exhales to expel air from the cavity formed by the face mask.

In some embodiments, the first sensor and the second sensor are removably coupled to the face mask such that the first sensor unit and the second sensor unit can be removed to wash the face mask.

Yet another implementation of the present disclosure is a face mask configured to be worn by a user. The face mask including a mask body formed of a flexible material and configured to completely cover the mouth and nose of the user such that a cavity is formed between the face of the user and the face mask, the mask body having an interior surface facing the mouth and nose of the user and an exterior surface opposite the interior surface, a first valve configured to open when the user inhales to allow air to enter the cavity formed by the face mask, the first valve including a first sensor for measuring a moisture level and an oxygen level of the air entering the cavity, and a second valve configured to open when the user exhales to expel air from the cavity formed by the face mask, the second valve including a second sensor for measuring a moisture level and an oxygen level of the air being expelled from the cavity, wherein both the first valve and the second valve are positioned on the mask body and extend from the interior surface to the exterior surface.

In some embodiments, the face mask further includes at least one light emitting diode (LED) positioned on one of the mask body, the first sensor unit, or the second sensor unit, and the at least one LED is configured to illuminate a first color if any of the moisture level of the air entering the cavity, the oxygen level of the air entering the cavity, the moisture level, or the oxygen level of the air being expelled from the cavity exceeds a threshold value or illuminate a second color if each of the moisture level of the air entering the cavity, the oxygen level of the air entering the cavity, the moisture level, and the oxygen level of the air being expelled from the cavity is below the threshold value.

In some embodiments, the first valve and the second valve are removably coupled to the mask body such that the first valve and the second valve can be removed to wash the face mask.

In some embodiments, the face mask further includes at least one short-range, ultra-high frequency (UHF) wireless transceiver configured to transmit data from the first sensor and the second sensor to a remote device.

In some embodiments, the remote device compares the moisture level and the oxygen level of the air entering the cavity to the moisture level and the oxygen level of the air being expelled from the cavity to determine whether a moisture level of the face mask or the user's oxygen saturation exceeds corresponding threshold values.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a diagram of an example face mask having integrated oxygen and moisture sensors, according to some embodiments.

FIG. 2 is a diagram of a sensor unit of the face mask of FIG. 1, according to some embodiments.

FIG. 3 is a block diagram of a control system for the face mask of FIG. 1, according to some embodiments.

FIG. 4 is a flow diagram of a process for detecting a user's oxygen saturation and excessive moisture while wearing the face mask of FIG. 1, according to some embodiments.

FIG. 5 is an example user interface for displaying detected oxygen saturation, moisture levels, and alerts to a user, according to some embodiments.

### DETAILED DESCRIPTION

Referring generally to the figures, a face mask having integrated moisture and oxygen sensors is shown, accordingly to various embodiments. The moisture and oxygen sensors may detect an amount of oxygen and moisture (e.g., humidity) in the air being inhaled and/or exhaled by a user (i.e., wearer) of the face mask. Based on the measured intake and/or output oxygen measurements, the user's blood oxygen saturation can be calculated. Similarly, using the intake and/or output moisture measurements, a moisture level of the mask can be determined. Subsequently, the user's blood oxygen saturation and/or the moisture level of the mask can be compared to threshold values (e.g., a minimum oxygen saturation value and/or a maximum moisture level) and, if either the oxygen saturation and/or the moisture level exceed a corresponding threshold, the user may be alerted (e.g., via a user interface).

In this manner, the face mask described herein may provide real-time feedback to the user both indicating the effectiveness of the face mask and reassuring the user that they are receiving an adequate oxygen supply. For example, the moisture level of the mask exceeding a maximum moisture level may indicate that the mask is retaining a high level of moisture (e.g., from the user's breath), making the mask less effective at filtering particulate and pathogens. Additionally, a moisture-logged mask may restrict the user's intake air supply, resulting in lower oxygen levels. Thus, by receiving alerts and/or feedback, the user may proactively remove and replace or wash the face mask. To this point, the face mask described herein may advantageously be formed of a washable material, such as double-knit cotton, nylon, polyester, polypropylene, etc., which allows the face mask to be reused. Additional features and advantages are described in greater detail below.

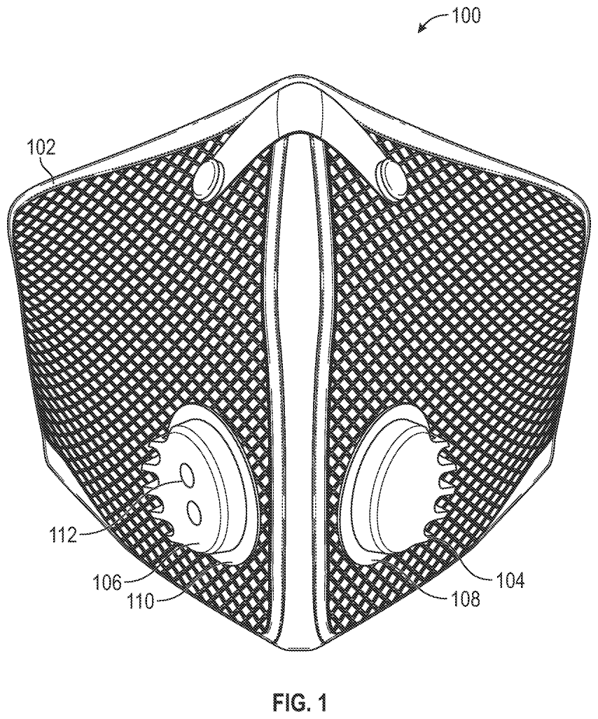

Turning first to FIG. 1, a diagram of an example face mask 100 having integrated oxygen and moisture sensors is shown, according to some embodiments. As mentioned above, face mask 100 is designed to be worn by a user (e.g., a human) to cover the user's nose and mouth in order to filter out particulate and/or pathogens when the user inhales, and/or to prevent the user from expelling particulate and/or pathogens (e.g., via saliva) when they exhale. Accordingly, face mask 100 is shown to include a mask body 102, which defines an interior surface that faces (i.e., is positioned closest to) the user's nose and mouth, and an exterior surface that is opposite from the interior surface. When properly worn, mask body 102 may form a cavity between the user's face (e.g., nose and mouth) and said interior surface; however, it will be appreciated that the edge of mask body 102 should fit snuggly against the user's skin to prevent air from leaking into the cavity when the user inhales and to prevent air from escaping form the edges of face mask 100 when the user exhales.

In general, mask body 102 is formed of a flexible material and, more specifically, a washable and flexible material. Non-limiting examples of the washable, flexible material used to form mask body 102 include non-woven fabrics, such as polypropylene, or woven fabrics such as felted wool, quilting cotton, nylon, polyester, silk, etc. In some embodiments, mask body 102 is formed of multiple layers of similar or different materials. For example, mask body 102 can include a cotton or polypropylene inner layer and a nylon or polyester outer layer. While not shown, one or more straps may be coupled to mask body 102 to allow the user to secure face mask 100 to their head. For example, at least one strap may extend from a first edge of mask body 102, around the user's head, and to a second edge of mask body 102. Additionally, in some embodiments, mask body may include a flexible metal or plastic nose piece which is preferably adjustable by the user (e.g., by bending) to allow the user to customize the fit of the nose piece around their nose, thereby further sealing the edges of mask body 102 against the user's skin.

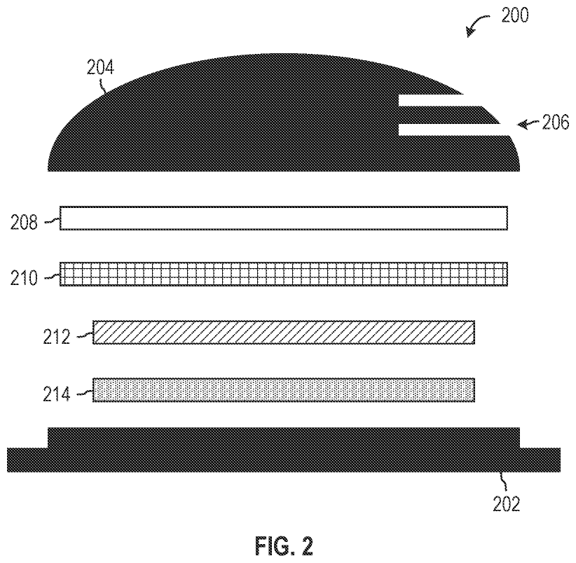

Coupled to mask body 102 are a first sensor unit 104 and a second sensor unit 106, which are described in detailed in FIG. 2. However, as a brief introduction, first sensor unit 104 and second sensor unit 106 may generally detect at least one of an oxygen content and a moisture level in air that is entering or exiting the cavity formed by face mask 100 against the user's nose and mouth. In some embodiments, first sensor unit 104 measures intake air (e.g., air that enters the cavity formed by face mask 100 as the user inhales) and second sensor unit 106 measures exhaust air (e.g., air that is expelled from the cavity formed by face mask 100 as the user exhales); however, it will be appreciated that the positioning and configuration of first sensor unit 104 and second sensor unit 106 may be altered in various embodiments. For example, in some cases, the positioning of first sensor unit 104 and second sensor unit 106 may be switched. In other embodiments, face mask 100 may only include a single, combined sensor unit that measures the oxygen content and moisture level of both intake and exhaust air. In yet other embodiments, face mask 100 may include additional oxygen and/or moisture sensors or may include separate oxygen and moisture sensors.

In some embodiments, first sensor unit 104 and/or second sensor unit 106 are housed in, or may include, a corresponding valve that prevents air from entering and/or exiting the cavity formed by face mask 100. Specifically, the valve(s) may be one-way valves that extend from the interior surface of mask body 102 to the exterior surface of mask body 102. For example, first sensor unit 104 may be positioned within a first valve or may include a first valve that only allows air flow into face mask 100 when the user inhales but that remains closed to prevent air from flowing out of face mask 100 when the user exhales. Correspondingly, second sensor unit 106 may be positioned within a second valve or may include a second valve that only allows air flow out of face mask 100 when the user exhales but that remains closed to prevent air from flowing into face mask 100 when the user inhales. Thus, the valve(s) of first sensor unit 104 and/or second sensor unit 106 may control the flow of air into, and out of, face mask 100.

In some embodiments, first sensor unit 104 and/or second sensor unit 106 (e.g., and/or their corresponding valves) are removably coupled to mask body 102. In other words, first sensor unit 104 and/or second sensor unit 106 may be removed (e.g., by a user) to allow mask body 102 to be replaced and/or washed. In some such embodiments, mask body 102 may include retaining elements 108, 110 to facilitate the removable coupling of first sensor unit 104 and/or second sensor unit 106. Retaining elements 108, 110 may include, for example, tabs, threading, ridges, gaskets, or any other components to removably couple first sensor unit 104 and/or second sensor unit 106 to mask body 102.

In some embodiments, face mask 100 can also include a user interface for, at least, presenting information to a user. In the example of FIG. 1, one or both of first sensor unit 104 and/or second sensor unit 106 can include indicator lights 112, which may be different or multi-colored light emitting diodes (LEDs); however, it will be appreciated that indicator lights 112 may be positioned on other parts of mask body 102. In some embodiments, indicator lights 112 include at least to different colored LEDs (e.g., green and red), although in other embodiments, indicator lights 112 include a single multi-colored LED. Indictor lights 112 may indicate to a user whether at least one of the user's oxygen saturation or the moisture level of the max exceeds a predefined threshold, as discussed in greater detail below. For example, if the moisture level of face mask 100 is too high (e.g., above a maximum level), a red LED may illuminate, whereas if the moisture level of face mask 100 is below the threshold (e.g., below the maximum level), a green LED may illuminate.

Referring now to FIG. 2, a diagram of a sensor unit 200 of face mask 100 is shown, according to some embodiments. In particular, FIG. 2 illustrates an “exploded” component view of sensor unit 200. Sensor unit 200 may be representative of, the same as, or functionally equivalent to first and second sensor units 104, 106, described above. However, for simplicity, a single sensor unit (e.g., one of sensor units 104, 106) is described in detail. It should also be appreciated that sensor unit 200 may be referred to herein as a valve assembly, particularly in certain embodiments where sensor unit 200 includes a one-way valve for preventing airflow in one direction (e.g., into or out of face mask 100), as described in detail below.

As shown, sensor unit 200 includes a two-piece housing formed of a lower housing 202 and an upper housing 204. Lower housing 202 and upper housing 204 may be formed of any suitable, rigid material, such as a plastic or metal. Lower housing 202 and upper housing 204 may be configured to be interlocked and may be either fixedly or removably coupled. For example, lower housing 202 may include threading (not shown) that interlocks with corresponding threading on upper housing 204 to couple the two components. In some embodiments, one of lower housing 202 or upper housing 204 may also include a seal (not shown) or gasket to prevent air from leaking at the area where lower housing 202 and upper housing 204 connected. As mentioned above, in other embodiments, lower housing 202 and upper housing 204 are fixedly coupled such that there is no joint or space between the two components.

In some embodiments, lower housing 202 is fixedly coupled to mask body 102, as described above. For example, lower housing 202 may be glued to, stitched to, or otherwise fastened to the fabric of mask body 102. In some embodiments, lower housing 202 may extend through mask body 102 from an interior surface to an exterior surface. For example, lower housing 202 main remain fixedly coupled to mask body 102 such that upper housing 204, and the components contained therein, can be removed. In other embodiments, lower housing 202 may be removably coupled to a separate fastening element (not shown) that is fixedly mounted to mask body 102 and that extends from the interior surface to the exterior surface of mask body 102. In some such embodiments, both lower housing 202 and upper housing 204, and the components contained therein, can be removed from mask body (e.g., to facilitate the washing of face mask 100).

In some embodiments, upper housing 204 includes one or more shaped openings 206 to allow air to flow through sensor unit 200. In FIG. 2, for example, shaped openings 206 are shown as generally rectangular slots; although, it should be appreciated that shaped openings 206 may include any shape and number of openings. For example, in another configuration, shaped openings 206 may include a plurality of circular openings formed on upper housing 204. In other embodiments, rather than shaped openings 206, upper housing 204 can include mesh, fabric, or another material that allows for air flow through sensor unit 200.

Looking now at the interior components of sensor unit 200, lower housing 202 and upper housing 204 are shown to enclose a valve 208 and/or a filter element 210; however, it will be appreciated that, in various different embodiments, sensor unit 200 may include only valve 208, only filter element 210, or both valve 208 and filter element 210. Additionally, it should be appreciated that the positioning of valve 208 and filter element 210 is not intended to be limiting. In some embodiments, valve 208 is formed of a flexible material, such as rubber, and is configured to allow air to flow in only one direction through sensor unit 200. For example, sensor unit 200 may be configured to sense only intake air (e.g., when a user inhales) and thus valve 208 may be configured to only allow air to flow into face mask 100 through sensor unit 200. In other words, valve 208 may remain closed, thereby blocking air flow through sensor unit 200, until the user either inhales or exhales, which can help to prevent particulate or pathogens from entering face mask 100 between breaths. Filter element 210 may be formed of any suitable filter material and is configured to filter out particulate and/or pathogens from the air entering or exiting sensor unit 200. For example, filter element 210 may be formed of a fabric such polypropylene. In some embodiments, filter element 210 is removable to allow for washing and/or replacement.

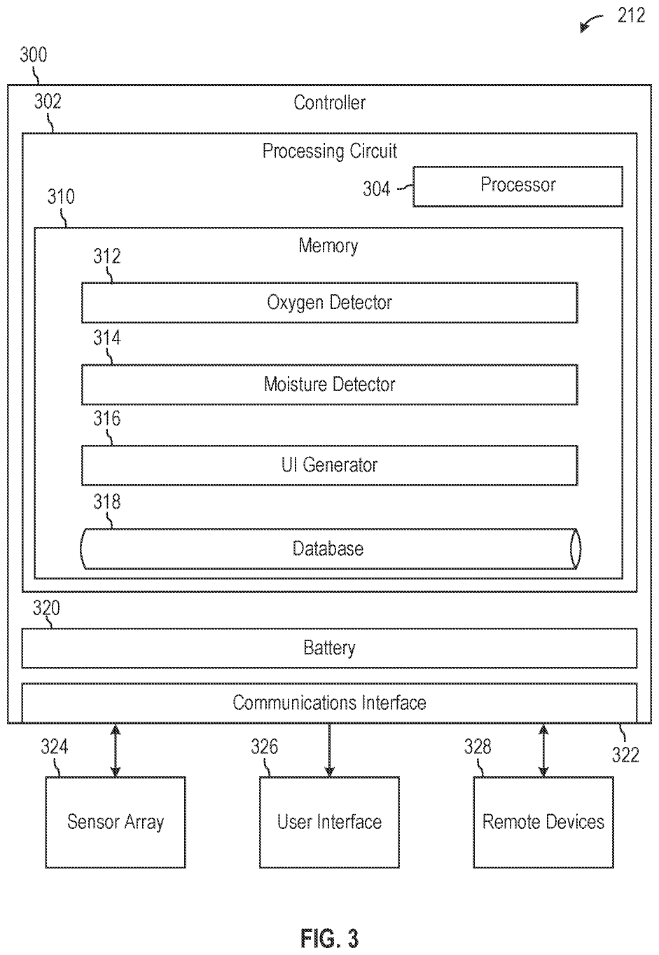

Still referring to FIG. 2, sensor unit 200 is also shown to include a control system 212 that includes one or more of a moisture sensor, an oxygen sensor, and a controller. Accordingly, system 212 may be configured to measure moisture and oxygen levels in the air that is passing through sensor unit 200 as the user inhales or exhales. For example, the air may flow over, around, or through system 212 where the moisture and oxygen levels are detected. In some embodiments, each of the moisture sensor, oxygen sensor, and/or controller are positioned on a single substrate, such as a printed circuit board (PCB) or flexible printed circuit (FPC). In other words, system 212 may be defined by a PCB or FCP that includes at least moisture and oxygen sensors and, in some cases, a controller. In other embodiments, rather than a controller, system 212 may include a wireless transceiver for transmitting data from the moisture and oxygen sensor(s) to a remote device. In some such embodiments, system 212 may include separate moisture and oxygen sensors, each include a wireless transceiver. In yet other embodiments, system 212 may include only a moisture sensor, an oxygen sensor, and at least one wireless transceiver. In such embodiments, the at least one wireless transceiver may transmit data from the moisture and oxygen sensor to a remote device. Additional features and description of system 212 are provided below with respect to FIG. 3.

In some embodiments, sensor unit 200 further includes a battery 214. Battery 214 may be any suitable type of replaceable and/or rechargeable battery. For example, battery 214 may be a button cell style battery. As shown, battery 214 may be a separate component from system 212 such that battery 214 may be removed and/or replaced, if necessary. In some embodiments, a battery holding element (not shown) may be mechanically and/or electrically coupled to system 212 such that battery 214 can provide energy to the components of system 212. In other embodiments, sensor unit 200 may include a separate battery holder and a separate electrical connection to system 212.

Referring now to FIG. 3, a block diagram illustrating system 212 in greater detail is shown, according to some embodiments. As shown, in some embodiments, system 212 may include a controller 300 for receiving data and/or processing from a sensor array 324 that includes at least one moisture sensor and at least one oxygen sensor. Controller 300 is shown to include a processing circuit 302 that includes a processor 304 and a memory 310. Processor 304 can be a general-purpose processor, an application specific integrated circuit (ASIC), one or more field programmable gate arrays (FPGAs), a group of processing components, or other suitable electronic processing components. In some embodiments, processor 304 is configured to execute program code stored on memory 310 to cause controller 300 to perform one or more operations.

Memory 310 can include one or more devices (e.g., memory units, memory devices, storage devices, etc.) for storing data and/or computer code for completing and/or facilitating the various processes described in the present disclosure. In some embodiments, memory 310 includes tangible, computer-readable media that stores code or instructions executable by processor 304. Tangible, computer-readable media refers to any media that is capable of providing data that causes the controller 300 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 310 can include random access memory (RAM), read-only memory (ROM), hard drive storage, temporary storage, non-volatile memory, flash memory, optical memory, or any other suitable memory for storing software objects and/or computer instructions. Memory 310 can include database components, object code components, script components, or any other type of information structure for supporting the various activities and information structures described in the present disclosure. Memory 310 can be communicably connected to processor 304, such as via processing circuit 302, and can include computer code for executing (e.g., by processor 304) one or more processes described herein.

While shown as individual components, it will be appreciated that processor 304 and/or memory 310 can be implemented using a variety of different types and quantities of processors and memory. For example, processor 304 may represent a single processing device or multiple processing devices. Similarly, memory 310 may represent a single memory device or multiple memory devices. Additionally, in some embodiments, controller 300 may be implemented within a single computing device (e.g., one server, one housing, etc.). In other embodiments controller 302 may be distributed across multiple servers or computers (e.g., that can exist in distributed locations). For example, controller 302 may include multiple distributed computing devices (e.g., multiple processors and/or memory devices) in communication with each other that collaborate to perform operations. Accordingly, it should be appreciated that, in certain embodiments, controller 300 may be a separate and/or remote component of system 212. For example, controller 300 may be hosted on a remote device (e.g., a user's smartphone or other computing device) and data may be wirelessly transmitted from sensor array 324 to controller 300 via a communication interface 322.

As described herein, communications interface 322 may facilitate communications between controller 300 and any external components or devices. For example, communications interface 322 can provide means for transmitting data to, or receiving data from, sensor array 324, a user interface 326, and/or remote devices 328, all of which are described in detail below. Accordingly, communications interface 322 can be or can include a wired or wireless communications interface (e.g., jacks, antennas, transmitters, receivers, transceivers, wire terminals, etc.) for conducting data communications. In some embodiments, communications interface 322 may also provide power to various external components. For example, controller 300 may power sensor array 324 via communications interface 322. In some such embodiments, power may be provided by a battery 320 of controller 300, which may be the same as or functionally equivalent to battery 214, described above. Further, controller 300 may also be powered by battery 320. Accordingly, battery 320 may be any suitable rechargeable and/or replaceable battery, such as a lithium-ion battery.

In various embodiments, communications via communications interface 322 may be direct (e.g., local wired or wireless communications) or via a network (e.g., a WAN, the Internet, a cellular network, etc.). For example, communications interface 322 can include a WiFi transceiver for communicating via a wireless communications network. In another example, communications interface 322 may include cellular or mobile phone communications transceivers. In yet another example, communications interface 322 may include a low-power or short-range wireless transceiver. Specifically, in some such embodiments, communications interface 322 may include a low-power ultra-high frequency (UHF) wireless transceiver (e.g., Bluetooth®).

Sensor array 324, as mentioned above, generally includes at least one moisture sensor, also referred to as a humidity sensor, and at least one oxygen sensor. The moisture sensor, for example, may be configured to measure a moisture level of the air passing over sensor array 324 (e.g., through sensor unit 200), which may be interpreted as a relative humidity level. In some embodiments, the moisture sensor may also, or alternatively, be configured to measure a level of moisture retained by the fabric of mask body 102. In some embodiments, the oxygen sensor is an electrochemical gas sensor or other similar sensor that can detect an amount of oxygen (e.g., in parts per million (ppm)) in the air passing through sensor unit 200. In some embodiments, sensor array 324 includes a wire grid or mesh configured to function as one or both of the moisture and temperature sensors.

User interface 326 can include any component or group of components that provide feedback to a user and/or that allow the user to interact with system 212. In some embodiments, user interface 326 includes one or more LEDs or lights that can be illuminated to provide feedback relating to humidity level, oxygen saturation, and the like. For example, user interface 326 may include at least one LED that illuminates red if the humidity level and/or oxygen saturation exceeds a threshold and green if the humidity level and/or oxygen saturation is within an acceptable range (e.g., below the threshold). In some embodiments, user interface 326 includes a display, such as an LED or LCD display. In some embodiments, user interface 326 includes an input device, such as a mouse, a keyboard, a keypad, a touchscreen, a touch surface, or the like.

Remote devices 328 may including any computing devices that are remote from controller 300. In some embodiments, remote devices 328 can include a memory (e.g., RAM, ROM, Flash memory, hard disk storage, etc.), a processor (e.g., a general-purpose processor, an application specific integrated circuit (ASIC), one or more field programmable gate arrays (FPGAs), a group of processing components, or other suitable electronic processing components), and a user interface (e.g., a touch screen), allowing a user to interact with controller 300. Remote devices 328 can include, for example, mobile phones, electronic tablets, laptops, desktop computers, workstations, and other types of electronic devices. In one example, a user's smartphone may be at least one of remote devices 328.

As mentioned above, it should be appreciated that, in some embodiments, controller 300 is remote from sensor array 324, and thereby sensor unit 200. For example, controller 300 may represent a user's smartphone or other computing device, such that only sensor array 324 is included in sensor unit 200. In such embodiments, sensor array 324 may wirelessly transmit (e.g., via a low-power, UHF wireless transceiver) data to controller 300 for processing. This configuration may utilize the computing power and larger battery or power source of the user's smartphone or other computing device to handle the processing and/or storage of data from sensor array 324. Accordingly, sensor unit 200 may be relatively small in size and may not require a large battery or large amounts of computing power. To this point, it should be appreciated that the following description of the functionality of controller 300 may equally apply to any computing device operated by a user (e.g., a smartphone running a software application). However, for simplicity, the processing and/or storage of data received from sensor array 324 is described herein with respect to controller 300.

Still referring to FIG. 3, memory 310 is shown to include an oxygen detector 312 configured to received and process data from the oxygen sensor of sensor array 324. In some embodiments, oxygen detector 312 may be configured to receive an electrical signal from the oxygen sensor of sensor array 324 and may convert the electrical signal to an oxygen level (e.g., in ppm). In other embodiments, oxygen detector 312 may receive a reading of the oxygen level directly from the oxygen sensor. In some embodiments, oxygen detector 312 receives a first reading from an intake oxygen sensor (e.g., in a first sensor unit 200, such as first sensor unit 104) and may store the first reading in a database 318. This first oxygen level reading may be a “baseline” value indicating the amount of oxygen in the air being inhaled by the user. Subsequently, when the user exhales, oxygen detector 312 may receive a second reading from an exhaust oxygen sensor (e.g., in a second sensor unit 200, such as second sensor unit 106) and may compare the stored first reading with the second reading to determine the amount of oxygen consumed or retained by the user. Accordingly, oxygen detector 312 may calculate the user's blood oxygen saturation by comparing a level of oxygen at the intake and exhaust units (e.g., first and second sensor units 104, 106) of face mask 100.

Memory 310 is also shown to include a moisture detector 314, also referred to as humidity detector 314, configured to received and process data from the moisture sensor of sensor array 324. In some embodiments, moisture detector 314 may be configured to receive an electrical signal from the moisture sensor of sensor array 324 and may convert the electrical signal to a moisture level (e.g., represented as a percentage out of 100% humidity/moisture). In other embodiments, moisture detector 314 may receive a reading of the moisture level directly from the moisture sensor. In some embodiments, moisture detector 314 receives a first reading from an intake moisture sensor (e.g., in a first sensor unit 200, such as first sensor unit 104) and may store the first reading in database 318. This first moisture level reading may be a “baseline” value indicating the amount of moisture in the air being inhaled by the user. Subsequently, when the user exhales, moisture detector 314 may receive a second reading from an exhaust moisture sensor (e.g., in a second sensor unit 200, such as second sensor unit 106) and may compare the stored first reading with the second reading to determine the amount of moisture retained by face mask 100. In some embodiments, face mask 100 may include only a single moisture sensor that directly measures the amount of moisture retained by face mask 100. In such embodiments, moisture detector 314 may simply receive and interpret the moisture level reading from the single moisture sensor.

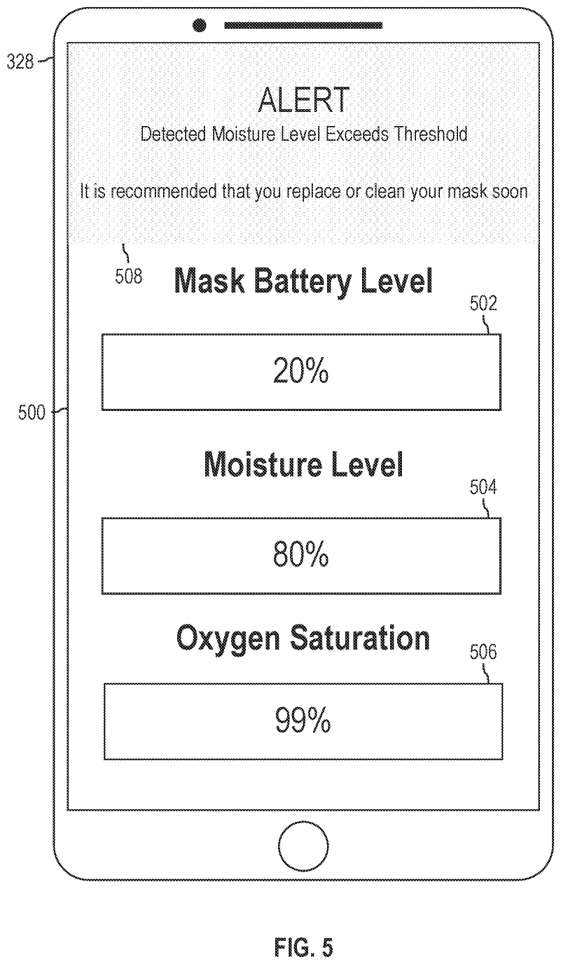

Memory 310 is also shown to include a user interface (UI) generator 316 configured to generate user interfaces based on oxygen and/or moisture data. For example, UI generator 316 may be configured to generate a graphical user interface that displays at least one of the user's calculated oxygen saturation, the moisture level of face mask 100, and/or notifications that indicate to the user whether their oxygen saturation is low or whether the moisture level of the mask is too high. One example of such a user interface is shown in FIG. 5, described below. In some embodiments, UI generator 316 may simply be configured to illuminate one or more indicator lights (e.g., indicator lights 112) responsive to the detected oxygen saturation and/or moisture level.

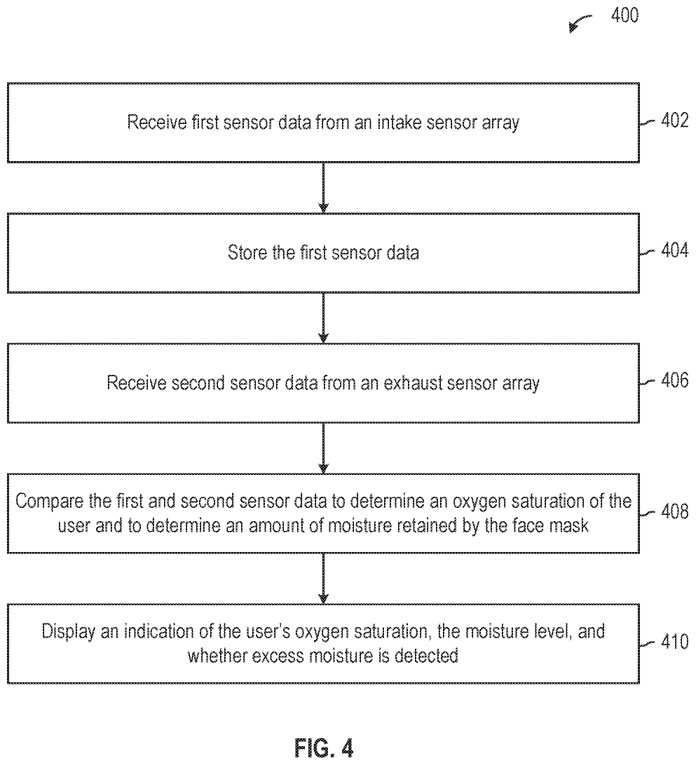

Referring now to FIG. 4, a flow diagram of a process 400 for detecting a user's oxygen saturation and excessive moisture while wearing face mask 100 is shown, according to some embodiments. As mentioned above, indications of the user's oxygen saturation and excessive moisture in face mask 100 can, advantageously, be utilized to provide real-time feedback to the user and/or to alert the user if their oxygen saturation is low or if the moisture level of the mask is too high. For example, if the user is alerted to either scenario, the user may be prompted to remove face mask 100 for a period of time and/or replace or wash face mask 100. In some embodiments, process 400 is implemented by controller 300, as described above. In other embodiments, process 400 is implemented by any computing device that receives data from sensor array 324, and more broadly sensor unit 200. It will be appreciated that certain steps of process 400 may be optional and, in some embodiments, process 400 may be implemented using less than all of the steps.

At step 402, first sensor data is received from an intake sensor array (e.g., first sensor unit 104). In general, the first sensor data may be received responsive to a user (e.g., a wearer of face mask 100) inhaling, which causes air to enter the intake sensor array. In some embodiments, the first sensor data includes at least an oxygen content measurement (e.g., in ppm) for the intake air. In some embodiments, the first sensor data further includes a moisture level measurement for the intake air. In some embodiments, the first sensor data is received as electrical signals that are then converted to digital data. In other embodiments, the first sensor data is digital data received directly from the intake sensor array (e.g., via a wireless connection). Subsequently, at step 404, the first sensor data may be stored in a database for later retrieval.

At step 406, second sensor data is received from an exhaust sensor array (e.g., second sensor unit 106). In general, the second sensor data may be received responsive to a user (e.g., a wearer of face mask 100) exhaling, which causes air to exit face mask 100 through the exhaust sensor array. In some embodiments, the second sensor data includes at least an oxygen content measurement (e.g., in ppm) for the exhaust air. In some embodiments, the second sensor data further includes a moisture level measurement for the exhaust air. In some embodiments, the second sensor data is received as electrical signals that are then converted to digital data. In other embodiments, the second sensor data is digital data received directly from the exhaust sensor array (e.g., via a wireless connection).

At step 408, the first and second sensor data is compared to determine an oxygen saturation of the user and to detect whether an amount of moisture retained by face mask 100. Specifically, the first and second recorded oxygen amounts may be used to calculate an oxygen saturation (i.e., blood oxygen saturation) of the user, which generally indicates the amount of oxygen-saturated hemoglobin in the user's blood relative to the total hemoglobin. For example, the first (i.e., stored) oxygen reading may be subtracted from the second oxygen reading to determine an amount of oxygen consumed or retained by the user. Based on this difference in oxygen readings, the user's oxygen saturation can be calculated.

Similarly, the first and second measured moisture levels may be used to determine the amount of moisture retained by face mask 100. For example, the first moisture level may be subtracted from the second moisture level to determine the amount of moisture retained by face mask 100. In other embodiments, the moisture emitted by the user (e.g., as detected by at least the exhaust sensor array) may be tracked over time. In such embodiments, the amount of moisture retained by face mask 100 with each of the user's breaths may be known (e.g., based on experimental data); thus, by tracking moisture over time, the amount of moisture retained by face mask 100 can be calculated. Alternatively, face mask 100 may include only a single moisture sensor that directly measures the amount of moisture retained in the fabric of face mask 100. In yet other embodiments, the first moisture level can indicate a relative humidity of the air in the environment or space occupied by the user. In some such embodiments, the first moisture level is compared to the second moisture level to determine an amount of moisture retained by face mask 100.

At step 410, a user interface that indicates at least one of the user's oxygen saturation, the moisture level of face mask 100, and whether excess moisture is detected is presented. In some embodiments, the user interface is a graphical user interface (e.g., as in FIG. 5) that displays an oxygen saturation level and a moisture level. In some embodiments, the oxygen saturation and moisture determined at step 408 may also be compared to predefined thresholds to determine whether the user's oxygen saturation is low, or the moisture retained by face mask 100 is high. For example, the oxygen saturation calculated at step 408 may be compared to a first threshold or multiple thresholds associated with safe oxygen saturation levels. If the calculated oxygen saturation falls below the threshold of, for example, 98% (e.g., which is the lower limit of “normal”), a notification may be presented to the user. The notification may include, for example, a push notification, an email, a text message, an automated phone call. In some embodiments, the notification causes one or more indicator lights positioned on face mask 100 (e.g., indicator lights 112) to illuminate to alert the user.

Similarly, the moisture level of the mask calculated at step 408 may be compared to a second threshold, below which face mask 100 is generally still effective at filtering particulate and/or pathogens. In some embodiments, the moisture level is compared to multiple thresholds associated with differing levels of effectiveness. If the moisture level of face mask 100 exceeds the threshold, a notification may be presented to the user. In some embodiments, the notification causes one or more indicator lights positioned on face mask 100 (e.g., indicator lights 112) to illuminate to alert the user that face mask 100 is retaining excessive moisture.

Referring now to FIG. 5, an example user interface 500 for displaying detected oxygen saturation, moisture levels, and alerts to a user is shown, according to some embodiments. As shown, interface 500 may be displayed on one of remote device 328, such as a user's smartphone. Additionally, or alternatively, interface 500 may be displayed on a user interface of controller 300 or system 212. As shown, interface 500 indicates a mask battery level 502 to the user. Mask battery level 502 may be determined based on, for example, data wirelessly received from one of sensor units 200 or controller 300 and may indicate to a user when sensor unit 200 and/or controller 300 should be charged or the battery replaced.

Additionally, interface 500 provides the user with an indication of a moisture level 504 and an oxygen saturation 506. As described above, moisture level 504 may indicate an amount of moisture retained by face mask 100. In some embodiments, moisture level 504 is displayed as a percentage of relative humidity. In other embodiments, moisture level 504 is displayed as a percentage of a maximum moisture level for face mask 100. In yet other embodiments, moisture level 504 may be presented as discrete levels (e.g., low, medium, high). Similarly, in some embodiments, oxygen saturation 506 can be displayed as a percentage. In other embodiments, oxygen saturation 506 is displayed as discrete levels (e.g., low, medium, high). Additionally, interface 500 is shown to include a notification 508 altering the user to a low oxygen saturation or a high moisture level. In this example, notification 508 is presented as a push notification that indicates that the moisture level of face mask 100 is too high (e.g., that the moisture level exceeds a maximum threshold).

### CONFIGURATION OF EXEMPLARY EMBODIMENTS

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

What is claimed is:

1. A face mask configured to be worn by a user, the face mask comprising:
a mask body formed of a flexible material and configured to cover the mouth and nose of the user such that a cavity is formed between the face of the user and the face mask, the mask body having an interior surface facing the mouth and nose of the user and an exterior surface opposite the interior surface;
a first sensor unit for measuring a moisture level and an oxygen level of the air entering the cavity;
a second sensor unit for measuring a moisture level and an oxygen level of the air being expelled from the cavity; and
at least one wireless transceiver configured to transmit data from the first sensor unit and the second sensor unit to a remote device.

2. The face mask of claim 1, further comprising at least one light emitting diode (LED) positioned on one of the mask body, the first sensor unit, or the second sensor unit, wherein the at least one LED is configured to:
illuminate a first color if any of the moisture level of the air entering the cavity, the oxygen level of the air entering the cavity, the moisture level, or the oxygen level of the air being expelled from the cavity exceeds a threshold value; or
illuminate a second color if each of the moisture level of the air entering the cavity, the oxygen level of the air entering the cavity, the moisture level, and the oxygen level of the air being expelled from the cavity is below the threshold value.

3. The face mask of claim 1, wherein the first sensor unit is housed in a first valve configured to open when the user inhales to allow air to enter the cavity formed by the face mask, the first valve extending from the interior surface to the exterior surface of the mask body.

4. The face mask of claim 1, wherein the second sensor unit is housed in a second valve configured to open when the user exhales to expel air from the cavity formed by the face mask, the second valve extending from the interior surface to the exterior surface of the mask body.

5. The face mask of claim 1, wherein the first sensor unit and the second sensor unit are removably coupled to the mask body such that the first sensor unit and the second sensor unit can be removed to wash the face mask.

6. The face mask of claim 1, wherein the remote device compares the moisture level and the oxygen level of the air entering the cavity to the moisture level and the oxygen level of the air being expelled from the cavity to determine whether a moisture level of the face mask or the user's oxygen saturation exceeds corresponding threshold values.

7. The face mask of claim 1, wherein the at least one wireless transceiver comprises a short-range, ultra-high frequency (UHF) transceiver.

8. The face mask of claim 1, wherein the flexible material comprises a washable, non-woven fabric or a plurality of washable, woven fabric layers.

9. A method comprising:
receiving first data from a first sensor positioned on a face mask configured to be worn by a user, the first sensor unit measuring a moisture level and an oxygen level of the air entering a cavity formed between the face of the user and the face mask as the user inhales;
receiving second data from a second sensor unit that measures a moisture level and an oxygen level of the air being expelled from the cavity as the user exhales;
comparing the first data and the second data to determine an oxygen saturation for the user and an amount of moisture retained by the face mask; and
providing a notification to the user if the oxygen saturation of the user exceeds a first threshold or if the amount of moisture retained by the face mask exceeds a second threshold.

10. The method of claim 10, wherein the oxygen saturation for the user is determined based on a difference between the oxygen level of the air entering the cavity and the oxygen level of the air being expelled from the cavity.

11. The method of claim 10, wherein the amount of moisture retained by the face mask is determined based on a difference between the moisture level of the air entering the cavity and the moisture level of the air being expelled from the cavity.

12. The method of claim 10, wherein providing the notification comprises one of:
illuminating a light emitting diode (LED) positioned on the face mask; or
presenting a graphical user interface via a user device associated with the user, wherein the graphical user interface indicates the notification and presents at least one of the first data or the second data.

13. The method of claim 10, wherein the first sensor is housed in a first valve positioned on the face mask and configured to open when the user inhales to allow air to enter the cavity formed by the face mask.

14. The method of claim 10, wherein the second sensor is housed in a second valve positioned on the face mask and configured to open when the user exhales to expel air from the cavity formed by the face mask.

15. The method of claim 10, wherein the first sensor and the second sensor are removably coupled to the face mask such that the first sensor unit and the second sensor unit can be removed to wash the face mask.

16. A face mask configured to be worn by a user, the face mask comprising:
a mask body formed of a flexible material and configured to completely cover the mouth and nose of the user such that a cavity is formed between the face of the user and the face mask, the mask body having an interior surface facing the mouth and nose of the user and an exterior surface opposite the interior surface;
a first valve configured to open when the user inhales to allow air to enter the cavity formed by the face mask, the first valve comprising a first sensor for measuring a moisture level and an oxygen level of the air entering the cavity; and
a second valve configured to open when the user exhales to expel air from the cavity formed by the face mask, the second valve comprising a second sensor for measuring a moisture level and an oxygen level of the air being expelled from the cavity, wherein both the first valve and the second valve are positioned on the mask body and extend from the interior surface to the exterior surface.

17. The face mask of claim 16, further comprising at least one light emitting diode (LED) positioned on one of the mask body, the first sensor unit, or the second sensor unit, wherein the at least one LED is configured to:
illuminate a first color if any of the moisture level of the air entering the cavity, the oxygen level of the air entering the cavity, the moisture level, or the oxygen level of the air being expelled from the cavity exceeds a threshold value; or
illuminate a second color if each of the moisture level of the air entering the cavity, the oxygen level of the air entering the cavity, the moisture level, and the oxygen level of the air being expelled from the cavity is below the threshold value.

18. The face mask of claim 16, wherein the first valve and the second valve are removably coupled to the mask body such that the first valve and the second valve can be removed to wash the face mask.

19. The face mask of claim 16, further comprising at least one short-range, ultra-high frequency (UHF) wireless transceiver configured to transmit data from the first sensor and the second sensor to a remote device.

20. The face mask of claim 19, wherein the remote device compares the moisture level and the oxygen level of the air entering the cavity to the moisture level and the oxygen level of the air being expelled from the cavity to determine whether a moisture level of the face mask or the user's oxygen saturation exceeds corresponding threshold values.
